(19) United States
(12) Patent Application Publication
(10) Pub. No.: US 2007/0015960 A1
(43) Pub. Date: Jan. 18, 2007

(54) Device for localizing, influencing and guiding of tracking bodies, and method for operating a marking device

(75) Inventors: Peter Gornert, Jena (DE); Jochen Heinrich, Naumburg (DE); Hendryk Richert, Jena (DE); Michael Roder, Hermsdorf (DE); Udo Warschewske, Berlin (DE)

Correspondence Address: MERCHANT & GOULD PC, P.O. BOX 2903, MINNEAPOLIS, MN, 55402-0903, US

(73) Assignee: Schaerer Mayfield Technologies GmbH, Walterdorf (DE)

(21) Appl. No.: 10/547,229

(22) PCT Filed: Jan. 19, 2004
(86) PCT No.: PCT/EP04/00366

§ 371 Date: Jun. 20, 2006

(30) Foreign Application Priority Data
Feb. 28, 2003 (DE) 10308965.9
Apr. 25, 2003 (DE) 10318849.5

(51) Int. Cl. A61B 1/00

(52) U.S. Cl. 600/102

(57) ABSTRACT

The invention relates to a device for localizing tracking bodies (10), comprising at least one tracking body which is placed inside a physiological structure, and a means which is situated outside of the structure and which consists of sensor clusters (20) in a sensor cluster arrangement (55) as well as a method for localizing and influencing the tracking body. The tracking body is provided in the form of a body which is characterized by a finite remanent magnetization with a variable magnetic dipole moment and an anisotropic magnetic dipole field resulting therefrom. The sensor clusters (20) are provided in the form of a plurality of gradiometer sensors (30) with a specific measuring geometry.

Optionally, physical/chemical properties and/or a trajectory of the tracking body may be altered in a specific manner by an externally acting magnetic field (H) and/or physiological processes in the environment of the at least one tracking body. In addition, it is possible to detect the location of a variable, in particular, a displaceable portion (57) which is associated with an expanded imaging means (60) of the sensor cluster arrangement (55) by means of a fixed portion (56) of the sensor cluster arrangement and to use the variable portion of the sensor cluster arrangement as a site marking in the expanded imaging arrangement.

The invention relates to a device for localizing, influencing, and guiding of tracking bodies and to a method for the operation of the marking device.

Marking devices in connection with tracking bodies are used in medical-biological applications for the specific marking of physiological structures or for marking of orientation points in operation fields. Such devices are often combined with imaging methods. The tracking bodies are configured in such a manner that their site and location within an operation field may unambiguously be identified by means of suitable examination methods. The travel resulting from the time sequence and the changes of the location of the tracking body in an organism is referred to as trajectory. This is recorded and analyzed, whereby it is possible to draw conclusions to functional sequences in an organ system which the tracking body has passed or is presently passing. An example is the swallowing of a capsule which shows an identifiable contrast in an X-ray image. From the location and the passing times of the contrast structure which is generated by the capsule in the X-ray image, conclusions may be drawn to the activity of the digestive tract and indigestions.

Such a method may i. a. be designed minimally invasive in that the tracking body emits the magnetic field of a magnetic dipole, which is measured. On the basis of the measuring data, the position of the magnetic dipole is determined. The magnetic dipole field is monitored and evaluated by means of magnetic field sensors which enable a resolution of a few nano Tesla. With a magnetic dipole specimen of approx. 0.08 Am², a site resolution of approx. 1 mm and an orientation resolution of approx. 0.1 angular degrees can be achieved in real time (distance sensor—marker approx. 15 cm). Such a tracking method does not need any additional energy supply for the activation of the tracking body and represents a negligible stress on the patient's organism.

The previously employed versions of such a tracking method are currently relatively limited in their range of applications. According to the state of the art, an inflexibly responding sensor construction is used which consists of an essentially rectangular plate which is arranged at a fixed distance from a patient lying below it. Such sensor constructions cover a more or less fixed predetermined spatial range. In connection with a tracking body with a fixed magnetization, the application possibilities of such a tracking method are limited essentially to the tracking of a passage of the tracking body through a given body lumen, e.g. the intestine.

Thus the object arises to advance the described method in such a manner that an increased flexibility of the tracking method by an improved sensor technology, accompanied by a higher measuring accuracy and a further miniaturization both of the sensor system and the tracking body may be achieved, whereby in particular possibilities for a specific and minimally invasive influencing of the tracking body from outside and an expanded functionality of the tracking body are to be created.

The object is solved by a device for the localization of tracking bodies with the characteristics of claim 1 and a method for the localization and influencing of at least one tracking body which is placed in a physiological environment, with the characteristics of claim 12, with the dependent claims including advantageous developments of the device and the method main claim.

According to the invention the marking device comprises a tracking body in the form of a body which is characterized by a finite remanent magnetization with a variable magnetic dipole moment and an anisotropic magnetic dipole field resulting therefrom. The sensor means according to the invention is designed in the form of a plurality of modular sensor clusters which are sensitive to the anisotropic dipole field and which cover a measuring range, with a plurality of gradiometer sensors being integrated in each sensor in a specific measuring geometry. A measuring and control unit is connected with the plurality of the sensor clusters.

Each sensor cluster represents one detector unit by means of which the location of the tracking body in space and its orientation with reference to the recorded magnetic dipole field may be determined. For this purpose, the individual sensor cluster with actually variable shape limitations comprises a plurality of gradiometers in a suitable mutual geometric arrangement. According to the invention, several sensor clusters are joined in such a manner that they cover a required examination field in an optimum manner. The resulting sensor cluster arrangement leads to some kind of “mosaic” of various sensor clusters, which may flexibly adapted to the shape of the patient's body and, in particular, be placed around it and thus advantageously covers a spatial range. The individual sensor clusters may be joined and connected for measuring in an essentially free manner. The measuring and control means monitors and controls the operation of the respective sensor cluster arrangement built in this manner.

The tracking body consists of a material with a remanent magnetization as high as possible and a coercive field strength as low as possible. The material of the tracking body therefore comprises an elongated magnetization hysteresis in the direction of the magnetization axis and a narrow magnetization hysteresis in the direction of the outer field strength. This ensures, on the one hand, that the magnetization of the tracking body is particularly high upon a shutdown of the outer magnetic field, a high magnetic dipole moment is generated, but, on the other hand, the magnetization may be cancelled by a relatively weak outer reversing magnetic field.

It is therefore appropriate to prefer tracking bodies from a neodym-iron-boron composition (NdFeB), AlNiCo, and various iron alloys which may be coated by a physiologically and magnetically neutral material.

The tracking body itself may be provided in two basic embodiments. In a first embodiment, it forms an integral part of a medical instrument, in particular, of a pointer means, of an endoscope or a similar medical probe means. In a second embodiment, it is configured as an object which is movable in an organism, in particular in body lumens.

In the first embodiment, the tracking body forms a pointer means which is linked with a corresponding instrument, which is detected by means of the sensor cluster arrangement with respect to its location and orientation. A great advantage of such a pointer means is that the detected measuring signal (the magnetic field strength of the dipole) is generated without the supply of energy in the form of an external excitation or any wiring and is detected in a simple manner. The exact location e.g. of an endoscope may be determined under these conditions with high accuracy outside the patient's body. In the second embodiment the tracking body moves freely within an implantation zone and serves as a self-contained probe for the physiological conditions prevailing therein, with an external influencing of same being possible, if required.

The tracking body comprises sections with properties which may be activated and/or with reactive, in particular, tissue-marking properties or properties releasing substances in a controlled manner and/or further similar properties which are sensitive to a given physiological environment and/or externally applied influences, in particular external magnetic fields. According to this development, the tracking body is formed as a carrier means for substances which are released in a certain physiological environment or due to a specific externally applied influence, in particular a magnetic field. This allows the transport of therapeutically or diagnostically effective substances to the action site and the controlled release.

The mentioned sensor cluster in a minimum configuration comprises a plurality of gradiometers for the localization of at least one tracking body, in particular, for the detection of its location in a three-dimensional coordinate system and its angular orientation. The single sensor cluster thus represents the smallest detector unit of the marking system.

For coupling the individual sensor clusters to a larger sensor cluster arrangement, they are provided with interfaces for the mutual connection with additional sensor clusters. This results either in a greater plurality of gradiometers which are distributed over at least two sensor clusters, or the sensor clusters interact as a network via exchanged control signals.

Sensor cluster arrangements are particularly advantageous which are designed as part of a patient support, such as e.g. a reclining bed, a head, arm, or back rest, a table top or similar means. Such sensor cluster arrangements may thus be implemented as “hidden” and increase the comfort for the patient and are integrated in a space-saving manner into an existing apparatus architecture. In each case, the sensor cluster arrangement covers an appropriate area of the examination field.

Two-piece embodiments of the sensor cluster arrangement are particularly advantageous. Such embodiments comprise a fixed portion and a variably arranged displaceable portion. The variable portion is arranged as a part for position marking of an external device, such as e.g. another diagnostic device as, for example, in the objective of a microscope. Here, the location of the variable portion of the sensor cluster arrangement is detected by the fixedly installed sensor cluster arrangement, and thus the site of the controlling external diagnostic device relative to the tracking body/sensor cluster arrangement system is exactly matched and adjusted.

In a method for the localization and influencing of at least one tracking body placed in a physiological environment its location in space and its orientation and/or its trajectory are determined by means of an arrangement of at least one plurality of gradiometer sensors which are combined to a sensor cluster from a measured distribution of a field strength and a field direction of the least one tracking body surrounded by a magnetic dipole field. Optionally, the determination of the position is combined with a specific influence and variation of physical/chemical properties of the tracking body or the trajectory of the tracking body by an externally acting magnetic field.

The tracking body is thereby localized with high accuracy in the examination zone, on the one hand, and, on the other hand, the possibility is created in conjunction with the high detection accuracy to induce influences of the physiological environment in hard to access implantation fields in a specific and minimally invasive manner in that the tracking body is influenced from the outside.

In a first embodiment of the method the tracking body is configured as a location reference point of a diagnostic probe means, in particular of a catheter or an endoscopic device, with a movement, a current location in space, and a current orientation of the location reference point of the probe means being continuously detected by the sensor cluster arrangement. This allows a minimally invasive tracking of such a medical instrument.

In another embodiment of the method the tracking body is placed as a freely movable indicator into the respective physiological environment, e.g. as a constituent of a suspension, with its movement, its current location ins space, and the current orientation of the indicator being continuously determined by the sensor cluster arrangement.

For the determination of the position of the tracking body measuring data for an amount and a direction of a certain vector of a magnetic field strength in each individual gradiometer sensor of the sensor cluster is obtained. This forms the initial data for an algorithm for a searching strategy for the localization of the tracking body, which is stored in a position determination means. The algorithm of the searching strategy executes procedures for an inverse tracking, in particular adaptive gradient procedures in combination with a fuzzy evolution algorithm.

When using several sensor clusters in a sensor cluster arrangement a dynamic integration of the sensor clusters in the sensor cluster arrangement is executed between the sensor clusters by means of an internal communication protocol, Thereby, in particular, the signal-noise ratio in the entire sensor cluster arrangement and the data quantity generated by the sensor cluster arrangement are optimized.

The external influencing of the tracking body is achieved in several ways. In a first embodiment the magnetic moment of the tracking body is influenced by means of an externally applied magnetic field in such a manner that its magnetization is changed and, in particular, cancelled. Thus, an optional activation and deactivation of the tracking body takes place.

In a further embodiment of the tracking body influencing the trajectory of the tracking body is actively shifted by means of an externally applied gradient field with a field gradient of a corresponding direction and the implanted tracking body or a tracking body made with a helical surface is moved into a corresponding direction in a rotating magnetic field.

All mentioned embodiments of the external tracking body influencing may be performed individually, but also in combination. They may further be combined with a specific release of substances which are bound by the tracking body. Due to the changed magnetization of the tracking body, changed binding properties are generated at its surface and surface-bound substances are released in a controlled manner. The combination of the mentioned methods enables to use the tracking body either for purely diagnostic purposes or as a pointer, but beyond this application also for a specific transport of an active substance within an implantation field and thereby to considerably expand the applicability of the tracking method.

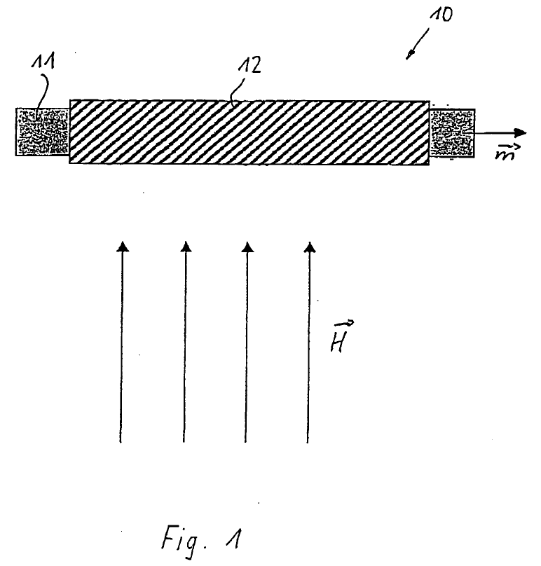

The inventive device and the inventive method will be described in more detail in the following with reference to embodiments. FIGS. 1 to 5 are used for illustrative purposes. For the same components or equally acting components, identical reference numerals will be used. In the drawings:

FIG. 1 is a schematic representation of a tracking body in an external magnetic field;

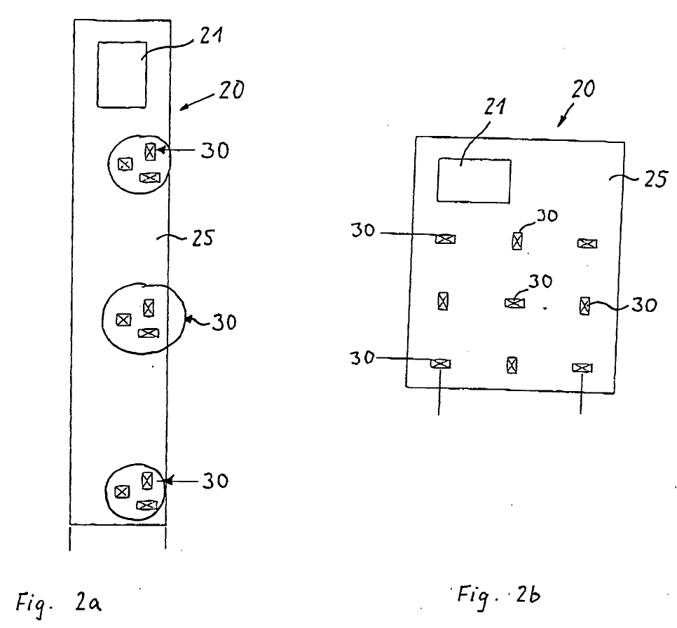

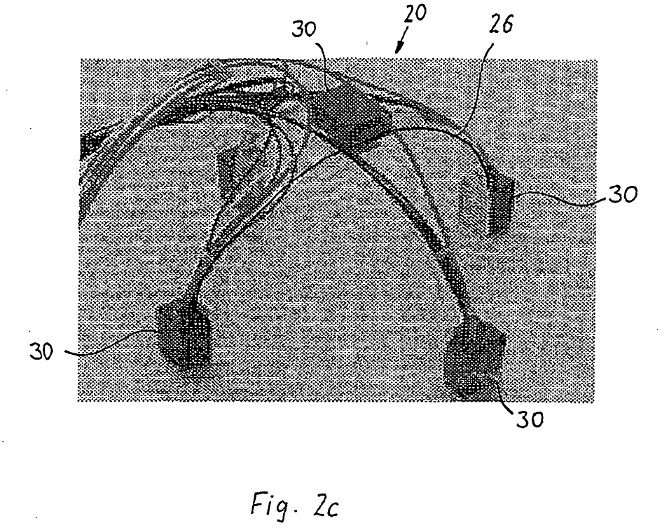

FIG. 2a is a schematic representation of a first embodiment of a sensor cluster;

FIG. 2b is a schematic representation of a second embodiment of a sensor cluster;

FIG. 2c is an exemplary illustration of a sensor cluster in a spatial spherical gradiometer geometry;

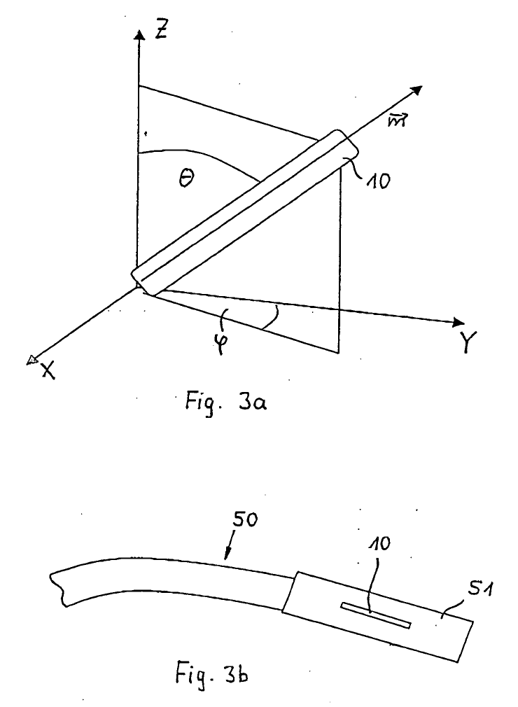

FIG. 3a is a schematic diagram of the location of a free tracking body In space with measured quantities to be determined;

FIG. 3b is a schematic illustration of a tracking body which is integrated in a probe head of an endoscopic instrument;

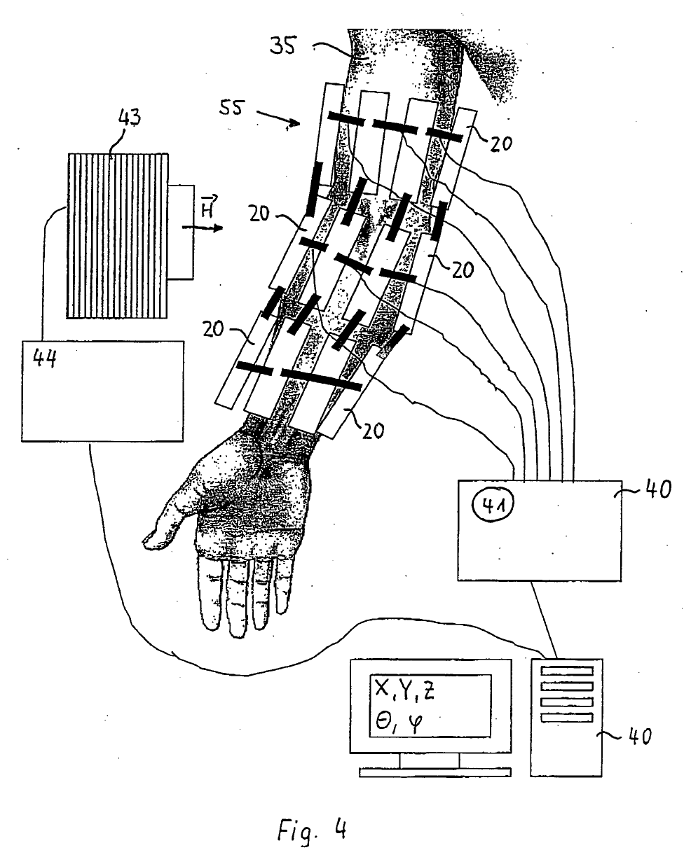

FIG. 4 is an exemplary arrangement of a sensor cluster, an external magnetic field coil, and a measuring and control means at a first examination object;

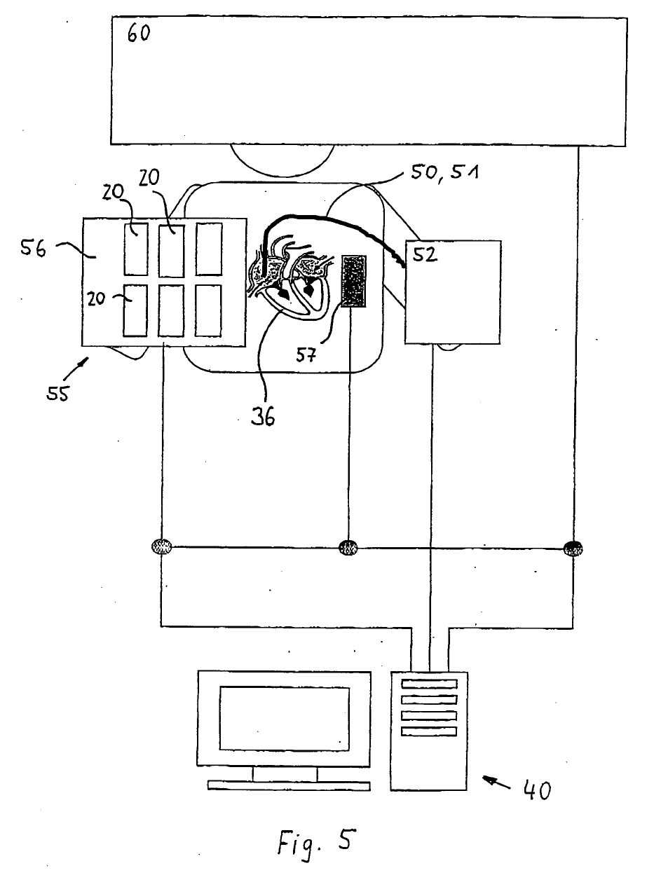

FIG. 5 is an exemplary arrangement of a fixed and a variable sensor cluster in combination with an endoscopic arrangement and a external means for computerized tomography for monitoring a cardiac catheter.

FIG. 1 shows an exemplary embodiment of an inventive tracking body 10. The tracking body consists of a magnetizable basic body 11 which on its surface comprises a sheath of an activatable portion 12. The basic body 11 is preferably made from a ferromagnetic material and suitably has a high remanent magnetization in combination with a coercive field strength to be achieved in the most simple manner by a given coil arrangement. Due to the finite remanence the basic body has a magnetic moment m, the location of which is determined in an external coordinate system.

By means of an externally applied magnetic field H the basic body is optionally magnetized or demagnetized, respectively, by the application of the coercive field strength, with the magnetic moment being able to be activated or cancelled as desired. Such a process is referred to in the following as hc switching. Due to the fact that the magnetic properties of the basic body are changed by the hc switching the interaction between the basic body 11 and the activatable portion 12 experiences a change, in particular with respect to specifically selected molecules. Under the influence of the hc switching, the activatable portion therefore either takes up molecules from the environment by absorption or absorption processes or releases previously bound molecules and substances into the immediate environment of the tracking body.

The tracking body aligns itself at a finite remanence under the influence of the external magnetic field H and its position is changed as a function of the external magnetic field. The free tracking body is therefore movable along a predefined trajectory under the influence of the external field. In conjunction with the hc switching process and the changed absorption capacity of the activatable portion, the possibility is given to utilize the tracking body as a carrier means for an active substance. For this purpose, the tracking body is first moved to and placed at an exactly defined site within the physiological environment under the influence of the external magnetic field H and then releases an active substance at an exactly defined time under the influence of the hc switching or takes up a substance from the environment, respectively.

The size of the tracking body which is typically used in the following embodiments may principally be varied within a large band width. Typical embodiments comprise a cylindrical shape of the basic body with a diameter of less than 1 mm and a length of less than 2 mm, with miniaturized embodiments with a diameter of less than 0.5 mm and a length of less than 1 mm being also conceivable. The size for concrete purposes which is actually used depends on the prevailing application conditions. In the selection, primarily the magnetic moment which can be achieved for a trouble-free detection and/or influencing of the magnetic body with a given dimensioning, the sensitivity of the external sensor devices and the actually prevailing physiological environmental condition, in particular the lumen size of vessels, cavities, and similar medical-biological parameters have to be considered. Finally, the dimensioning of the tracking body is also dependent on whether it is to be configured as a freely movable body or to be integrated in a medical instrument as a marking component.

Principally, a simultaneous employment of various types of tracking bodies and a distinguishing detection or a specific operation of these various types is possible in that tracking bodies 10 with basic bodies 11 are used, which comprise different magnetic characteristics, in particular with respect to the coercive field strength. Magnetic materials with a coercive field strength in the range from 0.1 kA/m to 500 kA/m are considered as being particularly suitable.

This makes it possible to activate a first type of tracking body by means of the hc switching method at a first coercive field strength of e.g. 500 kA/m with a finite remanence, to then reverse the direction of the external magnetic field and to cancel the magnetization of a second type with a coercive field strength of 0.1 kA/m, and to thereby selectively operate both the first and the second type, to use it as a means for transporting an active substance, or to detect either the first or the second type in its location. Thus, a specific influencing of an entire bandwidth of tracking bodies which are present at the same time in a given environment and are prepared for different tasks may be effected, while principally a wide type variety of a plurality of tracking bodies may be used at the same time. The tracking bodies may therefore remain on demand in the operation zone and be activated or deactivated, respectively, as required.

In the following, exemplary methods and devices for the location detection both for freely movable implanted tracking bodies and for tracking bodies which are formed as parts of an instrument will be described with reference to FIGS. 2a to 5.

A combination of the pure location detection and a trajectory tracking of the tracking bodies based thereon with the above described vehicle and indicator function is, however, always possible. With respect to the described hc switching a selective trajectory tracking of different tracking body types is possible as well.

FIGS. 2a to 2c show various embodiments of sensor clusters 20 which are used for the determination of location and orientation of the tracking bodies 10. One sensor cluster 20 represents the smallest detector unit by means of which a the location detection of the tracking body is effected. Various embodiments of the sensor clusters 20 may be used.

FIGS. 2a and 2b show plane geometries of plate-type sensor clusters 20 with a rather elongated rectangular shape in FIG. 2a and an essentially square embodiment in FIG. 2b. In these embodiments the individual sensor cluster consists of a base plate 25 each which is provided with a recess for attaching an interface means 21. A plurality of gradiometer sensors 30 is arranged on the base plate in a suitable geometrical arrangement. The embodiment of FIG. 2a shows an essentially linear gradiometer geometry for the tracking of a tracking body in a longitudinally extending operation field, FIG. 2c discloses a circular gradiometer geometry for the tracking of a tracking body trajectory in a plane, closely defined operation field, which enables a high site and location resolution.

The geometry of the gradiometer sensors 30 responds to the magnetic field strength vector which is generated by the magnetic dipole field of the tracking body 10 and registers its amount and direction at the respective site. The signals resulting therefrom are derived from the relevant sensor cluster 20 and evaluated in a measuring and control means, with the exact localization of the tracking body being calculated.

Apart from the plane embodiments of the sensor cluster 20 shown in FIGS. 2a and 2b, spatial measuring geometries of the gradiometers sensors 30 are possible as well. FIG. 2c shows such an arrangement in an exemplary illustration. The sensor cluster 20 in this case consists of an essentially spherical arrangement of gradiometers 30 which are stabilized in a defined measuring geometry by means of holding braces 26. The embodiment shown in FIG. 2c is particularly suited for the tracking of a trajectory of a tracking body in a volume zone.

For the determination of the location of the tracking body 10 an inverse tracking concept is employed. This means that in the method described in the following site and location parameters which uniquely characterize the tracking body are determined from the measured field distribution with the use of model adaptations, adaptive calculation methods, and the like.

FIG. 3a illustrates the parameters of the tracking body, which are to be determined. These are, on the one hand, the position within a given coordinate system, characterized by the coordinates X, Y, Z. Further, the magnetic dipole of the tracking body assumes an orientation in this point in the form of polar angular coordinates φ and θ. Another unknown quantity is the magnetic moment m of the tracking body. Thus, a maximum of six degrees of freedom is to be determined for the characterization of the tracking body. With a given number of gradiometers 30 in a sensor cluster 20, which is generally greater than 6, this results in the necessity of solving an overdetermined inverse problem. For the solution of this problem, adaptive gradient methods in combination with fuzzy evolution algorithms are employed, e.g. the Marquardt-Levenberg method, which in their entirety represent a searching strategy which is executed by the measuring and control unit.

The determination of the polar orientation is necessary, in particular, when the tracking body is configured as an integral part of an instrument, with the orientation of the magnetic dipole of the tracking body correlating with the location of the corresponding component of the instrument. FIG. 3b shows such an instrument schematically and by way of example. The figure shows a measuring head 51 of an endoscopic probe for the examination of a body cavity, e.g. a cardiac catheter or a gastro-intestinal probe. With such embodiments the position of the measuring head with respect to location and orientation is exactly determined by means of the mentioned methods. By means of the mentioned methods for inverse tracking and the method described in the following for an improved detection of the measuring signals, site resolutions of the spatial location of the tracking body can be achieved with an accuracy of 0.5 mm and orientation detections can be achieved with a resolution in the range of a few angular minutes, even under the influence of clinical contamination, high frequency fields in the environment, and the presence of metallic, but non-magnetic objects.

The sensitivity of the arrangement comprising tracking body and sensor cluster may be scaled by the magnitude of the magnetic moment of the tracking body. This is enabled, for example, in advance by the selection of a suitable tracking body. In addition, a continuous manipulation of the magnetic moment during tracking may be realized by the application of an external magnetic field, in particular, by the described hc switching process. Electromagnetic shielding means may essentially be omitted.

FIGS. 4 and 5 show exemplary applications of architectures of the sensor clusters 20 in a number of advantageous embodiments. FIG. 4 shows an architecture of sensor clusters 20 which are connected to a sensor cluster arrangement 55 and which are used in this embodiment for the tracking of a trajectory of a tracking body which has been implanted in the vessel system of an arm 35. The sensor cluster arrangement 55 is formed by a number of connections of the individual sensor clusters, which are indicated by fat connecting lines between the individual sensor clusters 20. The sensor cluster arrangement 55 encompasses the volume of the arm 35 at least partially. In this embodiment it is directly placed on the arm like a sleeve and is in contact with the skin. A simpler configuration of the sensor cluster arrangement, which is not shown herein is the design of a more or less rigid “tunnel” consisting of the sensor clusters 20, into which the arm is inserted. The tunnel may also be formed as part or an arm rest. The sensor cluster arrangement 55 which is required for the specific application is assembled for each case in a modular manner from the sensor clusters 20. The sensor clusters 20 thus form the basic “components” for the construction of a detector architecture which is adapted to special application purposes and which can be varied in virtually any way.

In the tracking of the trajectory of the tracking body within the vessel system of the arm 35, the sensor clusters 20 within the sensor cluster arrangement 55 form a mutually communicating network which is controlled and monitored by a measuring and control means 40. For this purpose, the measuring and control means includes a communication protocol for the interaction of the sensor clusters 20 within the network. The interaction serves at least for the self-calibration of the sensor cluster arrangement and the optimization of the signal-noise ratio of the measurement. To this end, the mutual location of the individual sensor clusters within the sensor cluster arrangement 55 is mutually adjusted in a given laboratory coordinate system and the geometric shape of the sensor cluster network obtained as a data structure. In another exemplary functionality of the communication protocol the most suitable sensor cluster 20 for the tracking of the trajectory is selected during the measurement, and the trajectory tracking is transferred from a first to a second sensor cluster so that the network of the sensor clusters 20 is continuously optimized. In conjunction therewith, operations for a noise filtering and an optimization of the measuring speed are performed. The measuring and control means 40 also comprises algorithms for finding global optima for the built-up configuration of the sensor cluster arrangement 55 and for error corrections. This contributes decisively to the stability of the measuring technique of the configuration.

The embodiment of FIG. 4 suitably also comprises a source for an external magnetic field for an above described Influencing of the tracking body or a plurality of tracking bodies in the form of hc switching or the active movement of the tracking body In the already described manner. In FIG. 4 this is symbolically indicated by a field coil 43 with a magnetic field control unit 44. It is advantageous to either design the field coil 43 movable along as many degrees of freedom as possible or to replace the single field coil by a field coil configuration for the generation of magnetic fields of various kinds, e.g. as gradient fields or circulating fields.

FIG. 5 shows in another exemplary embodiment an exemplary configuration of a tracking body integrated in a surgical instrument, which is arranged here in a measuring head 51 or a cardiac catheter 50. As in the embodiment of FIG. 4, the trajectory of the tracking body is tracked by a correspondingly designed sensor cluster arrangement 55. The sensor cluster arrangement in this special embodiment may be designed as a “tunnel” arranged around the upper part of the body or it may rest plane on the patient's chest. The most suitable design of the sensor cluster arrangement 55 is determined empirically for each case.

As a modification of the embodiment of the sensor cluster arrangement 55 from FIG. 4, a two-piece sensor cluster arrangement with a fixed portion 56 and a variable portion 57 is preferred in the embodiment according to FIG. 5. The fixed portion 56 is designed in the already described manner as a means for tracking the trajectory of the tracking body according to the embodiment of FIG. 4. The variable portion 57 forms a part of a controlling arrangement 60 for the execution of an imaging method, in particular for a computerized tomography or a magnetic resonance tomography, which spatially encompasses the sensor cluster arrangement. With such a configuration, the tracking of the measuring head 51 of the cardiac catheter is combined with an imaging method. The variable portion 57 of the sensor cluster arrangement thereby serves as a specimen both for the sensor cluster arrangement 55 itself and for the imaging arrangement 60. The position of the variable portion 57 is specified within the configuration of the sensor cluster arrangement 55 and is continuously tracked within the sensor cluster arrangement. It therefore forms a uniquely defined reference point in the coordinate system of the sensor cluster arrangement 55. On the other hand, the variable portion 57 of the sensor cluster arrangement is clearly outlined within the encompassing imaging arrangement 60, e.g. as a contrast image in a computerized tomography section or a magnetic resonance tomographic image.

The trajectory of the measuring head 51 with the integrated tracking body of the cardiac catheter 50 is thus detected with a very high accuracy by the sensor cluster arrangement 55. Thereby the location of the tracking body itself relative to the variable portion 57 of the sensor cluster arrangement is known so that a position representation of the measuring head 51 in the magnetic resonance tomographic image may be effected subsequently by means of suitable imaging means on the basis of the outlined contrast of the variable portion 57 of the sensor cluster arrangement 55. This may, for example, be done by a graphically inserted icon such as a stylized vector arrow which indicates the exact orientation of the measuring head for the surgeon. The variable portion 57 of the sensor cluster arrangement 55 therefore functions as a kind of “magnifying glass” or microscope for a local improvement of the image resolution of the magnetic resonance tomographic image or an image obtained through another method from the arrangement 60 so that the surgeon may manipulate the cardiac catheter at a cardiac catheter control unit 52 in a particularly exact and precise manner.

### LIST OF REFERENCE NUMERALS

10 Tracking body
11 Basic body, ferromagnetic
12 Activatable portion
20 Sensor cluster
21 Interface
25 Base plate
26 Holding braces
30 Gradiometer arrangement
35 Arm
36 Heart
40 Measuring and control means
41 Communication protocol
43 Magnetic field coil
44 Magnetic field control unit
50 Probe, cardiac catheter
51 Probe head
52 Probe control unit
55 Sensor cluster arrangement
56 Sensor cluster arrangement, fixed portion
57 Sensor cluster arrangement, variable portion
60 External imaging arrangement
m Magnetic dipole moment
X, Y, Z Coordinates
φ, θ Orientation angle

1. Device for localizing, influencing, and guiding of tracking bodies, comprising
at least one tracking body placed inside a physiological structure, and a sensor situated outside of the physiological structure for the determination of the location of the tracking body, as well as a measuring and control unit,
at least one tracking body having a finite remanent magnetization with a variable magnetic dipole moment and an anisotropic magnetic dipole field resulting therefrom,
wherein the sensor comprises a plurality of modular sensor clusters sensitive to the anisotropic dipole field and covering a measuring range, with one each at least one of a plurality of magnetic field sensors being integrated in the sensor cluster in a specific measuring geometry,
the measuring and control unit being linked for measuring with the plurality of the sensor clusters.

2. The device according to claim 1, wherein the at least one tracking body comprises a material with a remanent magnetization as high as possible and with a coercive field strength as low as possible.

3. The device according to claim 2, wherein the at least one tracking body comprises a neodym-iron-boron composition, NdFeB, which is coated by a physiologically and magnetically neutral material.

4. The device according to claim 1, wherein the at least one tracking body forms an integral part of a medical instrument comprising a medical pointer, an endoscope, or similar other probe.

5. The device according to claim 1, wherein the at least one tracking body is configured as a freely movable object which circulates in an organism, in particular in body cavities.

6. The device according to claim 5, wherein the at least one tracking body comprises activatable and/or reactive, in particular, tissue marking portions which release substances in a controlled manner and/or similar constituents sensitive to a given physiological environment, external magnetic fields.

7. The device according to claim 1, wherein each individual sensor cluster comprises a minimum configuration of a plurality of gradiometers for a localization of the at least one tracking body, for a detection of a location in space and a detection of an orientation.

8. The device according to claim 1, wherein the individual sensor cluster comprises interfaces for the connection with a plurality of further sensor clusters.

9. The device according to claim 1, wherein the sensor cluster comprises part of a patient support, in particular as a part of head, arm, or back rests, table tops or similar means.

10. The device according to claim 1, wherein a plurality of interconnected sensor clusters forms a sensor cluster arrangement, with the sensor cluster arrangement covering an appropriate zone of the examination field.

11. The device according to claim 1, wherein the plurality of sensor clusters comprises a fixed portion and a variable, displaceable portion, with the variable portion being designed as comprising a part for position marking in an external imaging arrangement, in particular, in a magnetic resonance tomography or computerized tomography.

12. A method for localizing and influencing of a tracking body which is placed into a physiological environment, wherein
from a measured distribution of the field strength and the field direction of at least one tracking body surrounded by a magnetic dipole field, its position in space and its orientation and/or its trajectory are determined by an arrangement of at least one of a plurality of magnetic field sensors combined to a sensor cluster, and/or
optionally, physical/chemical properties and/or a trajectory of the at least one tracking body are altered in a specific manner by an externally acting magnetic field and/or physiological processes in the environment of the at least one tracking body.

13. The method according to claim 12, wherein the tracking body comprises a location reference point of a diagnostic probe and/or sensor means, in particular, of a catheter or an endoscopic device, with a movement, a current location in space, and a current orientation of the location reference point being continuously determined.

14. The method according to claim 12, wherein the tracking body as a freely movable indicator is implanted into the respective physiological environment, as an diagnostically active constituent of a suspension, with a movement, a current location in space, and a current orientation of the indicator being continuously determined by the sensor cluster arrangement.

15. The method according to claim 12, wherein the determination of the location of the tracking body is effected from measuring data via an amount and a direction of a certain vector in each individual magnetic field sensor of the sensor cluster of a magnetic field strength of the magnetic field generated by the tracking body, with initial data for an algorithm for a searching strategy for the localization of the tracking body being stored in a position determination means.

16. The method according to claim 15, wherein the algorithm for the searching strategy executes procedures for an inverse tracking, in particular adaptive gradient procedures in combination with a fuzzy evolution algorithm.

17. The method according to claim 12, wherein when using several sensor clusters a dynamic integration of the sensor clusters in the sensor cluster arrangement is performed between the sensor clusters by means of an internal communication protocol, with an optimization of the signal-noise ratio in the entire sensor cluster arrangement and an optimization of the data quantity generated by the sensor cluster arrangement being effected.

18. The method according to claim 12, wherein the magnetic moment of the at least one tracking body is influenced by externally applied magnetic field wherein its magnetization is changed, in particular, cancelled, or generated.

19. The method according to claim 12, wherein an active displacement of the at least one implanted, freely movable tracking body is effected by an externally applied magnetic field with a field vector oriented in a corresponding direction.

20. The method according to claim 12, wherein a controlled release of substances bound on the surface of the tracking body is effected by the changed magnetization of the at least one tracking body.

21. The method according to claim 12, wherein the location of a variable displaceable portion which is associated with an expanded sensor cluster arrangement is detected by a fixed portion of the sensor cluster arrangement and/or the variable portion of the sensor cluster arrangement is used as a location marker in the expanded imaging arrangement, with the location in space and the orientation of the tracking body which are determined by the sensor cluster arrangement being inserted into the generated image.
